SEQUENCE LISTING (11 sequences)

SEQ ID NO: 1 - DNA, 518 bp, Canis lupus familiaris
  Interleukin 2 (1)..(518)
agctctcccatatggtcgacctgcaggcggccgcactagtgattggtaccatgtacaaaatgcaactcttgtcttgcatcgcactgacgcttgtacttgtcgcaaacagtgcacctattacttcaagctctacaaaggaaacagagcaacagatggagcaattactgctggatttacagttgcttttgaatggagttaataattatgagaacccccaactctccaggatgctcacatttaagttttacacgcccaagaaggccacagaatttacacaccttcaatgtctagcagaagaactcaaaaacctggaggaagtgctaggtttacctcaaagcaaaaacgttcacttgacagacaccaaggaattaatcagcaatatgaatgtaacacttctgaaactaaagggatctgaaacaagttacaactgtgaatatgatgacgagacagcaaccattacagaatttctgaacaaatggattaccttttgtcaaagcatcttctcaacactgacttga

SEQ ID NO: 2 - DNA, 17 bp, artificial sequence
  Primer for amplification IL-2 gene
  Primer forward (1)..(17)
tgatgagtgcattggag

SEQ ID NO: 3 - DNA, 18 bp, artificial sequence
  Primer for amplification IL2
  primer reverse (1)..(18)
gattctttttgtaagccc

SEQ ID NO: 4 - DNA, 1611 bp, canis lupus familiaris
  DNA-sequence coding for fusionproteine interleukin 12 (1)..(1611): DNA-sequence coding for fusionproteine comprising the first 987 nucleotids from p40 cDNA of IL12, then 30 nucleotides coding for 10 aminoacids from bovine elastin and then the nucleotides of p35 cDNA coding for IL12.
  ref <301>: AUTHORS: Buttner M., Belke-Louis G., Rziha H-J., McInnes C., Kaaden O-R
  ref <302>: TITLE: DETECTION, cDNA CLONING AND SEQUENCING OF CANINE INTERLEUKIN 12
  ref <303>: JOURNAL: Cytokine
  ref <304>: VOLUME: 10
  ref <305>: ISSUE: 4
  ref <306>: PAGES: 241-248
  ref <307>: DATE: 1998-01-01
  ref <308>: DATABASE ACCESSION NUMBER: U49100
  ref <309>: DATABASE ENTRY DATE: 1996-01-01
  ref <313>: RELEVANT RESIDUES IN SEQ ID NO: (1)..(1611)
atgcatcctcagcagttggtcatctcctggttttccctcgttttgctggcgtcttccctcatgaccatatgggaactggagaaagatgtttatgttgtagagttggactggcaccctgatgcccccggagaaatggtggtcctcacctgccatacccctgaagaagatgacatcacttggacctcagcgcagagcagtgaagtcctaggttctggtaaaactctgaccatccaagtcaaagaatttggagatgctggccagtatacctgccataaaggaggcaaggttctgagccgctcactcctgttgattcacaaaaaagaagatggaatttggtccactgatatcttaaaggaacagaaagaatccaaaaataagatctttctgaaatgtgaggcaaagaattattctggacgtttcacatgctggtggctgacggcaatcagtactgatttgaaattcagtgtcaaaagtagcagaggcttctctgacccccaaggggtgacatgtggagcagtgacactttcagcagagagggtcagagtggacaacagggattataagaagtacacagtggagtgtcaggaaggcagtgcctgcccctctgccgaggagagcctacccatcgaggtcgtggtggatgctattcacaagctcaagtatgaaaactacaccagcagcttcttcatcagagacatcatcaaaccagacccacccacaaacctgcagctgaagccattgaaaaattctcggcacgtggaggtcagctgggaataccccgacacctggagcaccccacattcctacttctccctgacattttgcgtacaggcccagggcaagaacaatagagaaaagaaagatagactctgcgtggacaagacctcagccaaggtcgtgtgccacaaggatgccaagatccgcgtgcaagcccgagaccgctactatagttcatcctggagcgactgggcatctgtgtcctgcagtgtcccgggagtcggtgtcccaggtgtcggtaggagcctccccacagcctcaccgagcccaggaatattccagtgcctcaaccactcccaaaacctgctgagagccgtcagcaacacgcttcagaaggccagacaaactctagattatattccctgcacttccgaagagattgatcatgaagatatcacaaaggataaaaccagcacagtggaggcctgcttaccactggaattaaccatgaatgagagttgcctggcttccagagagatctctttgataactaacgggagttgcctggcctctggaaaggcctcttttatgacggtcctgtgccttagcagcatctatgaggacttgaagatgtaccagatggaattcaaggccatgaacgcaaagcttttaatggatcccaagaggcagatctttctggatcaaaacatgttgacagctatcgatgagctgttacaggccctgaatttcaacagtgtgactgtgccacagaaatcctcccttgaagagccggatttttataaaactaaaatcaagctctgcatacttcttcatgctttcagaattcgtgcggtgaccatcgatagaatgatgagttatctgaattcttcctaa

SEQ ID NO: 5 - DNA, 27 bp, artificial sequence
  Primer for amplification p40 of IL12
ggtaccatgcatcctcagcagttggtc

SEQ ID NO: 6 - DNA, 50 bp, artificial sequence
  Primer for amplification p40 of IL12
cgtctcaggacaccgactcccgggacactgcaggacacagatgcccagtc

SEQ ID NO: 7 - DNA, 44 bp, artificial sequence
  Primer for amplification p35 of IL12
cgtctcggtcccaggtgtcggtatgtgcccgccgcgcggcctcc

SEQ ID NO: 8 - DNA, 50 bp, artificial sequence
  Primer for amplification p35 of IL12
acgcgtttaggaagaattcagataactcatcattctatcgatggtcaccg

SEQ ID NO: 9 - DNA, 669 bp, Canis lupus familiaris
  Interleukin IL12A (1)..(669): natural killer cell stimulatory factor 1, cytotoxic lymphocyte maturation factor 1, p35) (IL12A)
atgtgcccgccgcgcggcctcctccttgtgaccatcctggtcctgctaagccacctggaccaccttacttgggccaggagcctccccacagcctcaccgagcccaggaatattccagtgcctcaaccactcccaaaacctgctgagagccgtcagcaacacgcttcagaaggccagacaaactctagattatattccctgcacttccgaagagattgatcatgaagatatcacaaaggataaaaccagcacagtggaggcctgcttaccactggaattaaccatgaatgagagttgcctggcttccagagagatctctttgataactaacgggagttgcctggcctctggaaaggcctcttttatgacggtcctgtgccttagcagcatctatgaggacttgaagatgtaccagatggaattcaaggccatgaacgcaaagcttttaatggatcccaagaggcagatctttctggatcaaaacatgttgacagctatcgatgagctgttacaggccctgaatttcaacagtgtgactgtgccacagaaatcctcccttgaagagccggatttttataaaactaaaatcaagctctgcatacttcttcatgctttcagaattcgtgcggtgaccatcgatagaatgatgagttatctgaattcttcctaa

SEQ ID NO: 10 - DNA, 1015 bp, Canis lupus familiaris
  Interleukin IL12A (1)..(1015): natural killer cell stimulatory factor 1, cytotoxic lymphocyte maturation factor 1, p40) (IL12A)
  ref <301>: AUTHORS: Buttner M, Belke-Louis G, Rziha HJ, McInnes C, Kaaden OR
  ref <302>: TITLE: Detection, cDNA cloning and sequencing of canine interleukin 12
  ref <303>: JOURNAL: Cytokine
  ref <304>: VOLUME: 10
  ref <305>: ISSUE: 4
  ref <306>: PAGES: 241-248
  ref <307>: DATE: 1998-04-01
  ref <313>: RELEVANT RESIDUES IN SEQ ID NO: (1)..(1015)
atgcatcctcagcagttggtcatctcctggttttccctcgttttgctggcgtcttccctcatgaccatatgggaactggagaaagatgtttatgttgtagagttggactggcaccctgatgcccccggagaaatggtggtcctcacctgccatacccctgaagaagatgacatcacttggacctcagcgcagagcagtgaagtcctaggttctggtaaaactctgaccatccaagtcaaagaatttggagatgctggccagtatacctgccataaaggaggcaaggttctgagccgctcactcctgttgattcacaaaaaagaagatggaatttggtccactgatatcttaaaggaacagaaagaatccaaaaataagatctttctgaaatgtgaggcaaagaattattctggacgtttcacatgctggtggctgacggcaatcagtactgatttgaaattcagtgtcaaaagtagcagaggcttctctgacccccaaggggtgacatgtggagcagtgacactttcagcagagagggtcagagtggacaacagggattataagaagtacacagtggagtgtcaggaaggcagtgcctgcccctctgccgaggagagcctacccatcgaggtcgtggtggatgctattcacaagctcaagtatgaaaactacaccagcagcttcttcatcagagacatcatcaaaccagacccacccacaaacctgcagctgaagccattgaaaaattctcggcacgtggaggtcagctgggaataccccgacacctggagcaccccacattcctacttctccctgacattttgcgtacaggcccagggcaagaacaatagagaaaagaaagatagactctgcgtggacaagacctcagccaaggtcgtgtgccacaaggatgccaagatccgcgtgcaagcccgagaccgctactatagttcatcctggagcgactgggcatctgtgtcctgcagttaggttccacccccaggatgaatcttgg

SEQ ID NO: 11 - DNA, 30 bp, bovis spec.
  Elastin Motif (1)..(30)
  ref <301>: AUTHORS: Anderson R, Macdonald I, Corbett T, Hacking G, Lowdell MW, Prentice HG
  ref <303>: JOURNAL: Hum Gene Ther.
  ref <304>: VOLUME: 8
  ref <305>: ISSUE: 9
  ref <306>: PAGES: 1125-1135
  ref <307>: DATE: 1997-06-10
  ref <313>: RELEVANT RESIDUES IN SEQ ID NO: (1)..(30)
gttcctggagtaggggtacctggagtgggc

(19) United States
(12) Patent Application Publication
(10) Pub. No.: US 2010/0279414 A1
(43) Pub. Date: Nov. 4, 2010

(54) INVENTION CONCERNING THE EXPRESSION AND PERMANENT RELEASE OF CANINE INTERLEUKINES

(76) Inventors: Eberhard H. Burkhardt, Pohlheim-Watzenborn (DE); Vladimir Kocoski, Wurzburg (DE); Sandra Preis, Pohlheim-Hausen (DE); Norbert Tautz, Lubeck (DE)

Correspondence Address: CLARK & BRODY, 1700 Diagonal Road, Suite 510, Alexandria, VA, 22314, US

(21) Appl. No.: 12/668,904

(22) PCT Filed: Jul. 12, 2008
(86) PCT No.: PCT/DE2008/001133

§ 371 Date: Jun. 21, 2010

(30) Foreign Application Priority Data
Jul. 13, 2007 (DE) 1020070330725

(51) Int. Cl. C12N 15/09; C12N 5/10

(52) U.S. Cl. 435/455; 435/352; 435/347

(57) ABSTRACT

The present invention concerns the production, specific expression, and permanent release of canine interleukines, especially interleukin-2 (cIL-2) or interleukin-12 (cIL-12), from transfected BHK Tet-On cells, as well as the utilization of lymphokin-activated killer cells which were generated therewith for the tumor therapy in dogs.

The present invention relates to a procedure for the specific expression and permanent release of canine interleukines, in particular interleukin-2 (cIL-2) or interleukin-12 (cIL-12), from transfected BHK Tet-On cells.

These cells continuously produce interleukin-2 and interleukin-12, respectively, and are employed for the permanent stimulation of lymphokin-activated killer cells (LAK) for the production of a medication which is suitable for use in tumor therapy, especially in dogs.

### STATE OF THE ART

Due to the poor effectivity of classical tumor therapies in some tumor patients (surgical removal of the tumor, radiation and chemotherapy) with respect to the complete removal of tumor metastases and residual tumor cells, various components of the immune system which the body itself also uses against the tumor provide an opportunity to fight the tumor specifically and at the single-cell level. All approaches employing components of the immune system fall under the category of the so-called immune therapy. On of these approaches is the use of cytotoxic lymphocytes (a subpopulation of T cells and NK cells), which are prestimulated with cytokines enhancing their cytolytic and antitumoral effect and then injected into the patient.

It is well known from the literature and own scientific results that in dogs, interleukin-2 and interleukin-12 exhibit numerous anti-tumoral effects, e.g. an enhanced cytolytic effect via molecules which cause necrosis or apoptosis in tumor cells, and in addition act synergistically with T- and NK cells. Interleukin-2 and interleukin-12 alone or in combination are therefore particularly suitable for the production of a medication for the tumor therapy.

The state of the art for the production of human interleukin 2 in eukaryontic cells is known from JP62032885, in which the human IL-2 gene is expressed together with a PvuII fragment of the bovine papilloma virus. The recombinant synthesis of interleukin 12 from human blood cells is known from CN1373221. CN 1347622 reveals a procedure for the recombinant synthesis of human interleukin-12 in insect virus strains.

Disadvantageous is however that these recombinantly produced interleukines require further purification steps prior to an application in the patient in order to be free of viral or bacterial cells. Furthermore, they have to be administered externally to the tumor cells or the patient, leading to in- and decreasing cytokine concentrations. Severe side effects may therefore be associated with systemic medication of the patient. A target-oriented, approximately constant and thus physiological cytokine concentration is absolutely desirable. Further disadvantages are based on the fact that these cytokines are of human origin. Since the therapy will be carried out in dogs, canine interleukines as allogenic proteins are to be preferred. An involvement of xenogenic proteins should be avoided to achieve physiological conditions as far as possible.

For the tumor therapy in dogs, US 2007053873 reveals a procedure for the preparation and activation of canine lymphocytes in the presence of anti-canine CD3 antibodies and human interleukin-2 (IL-2), as well as for the treatment of tumors in dogs. Thereby it is of disadvantage that the use of CD3 antibodies is time consuming and expensive, and also severe side effects caused by the additional antibodies cannot be excluded. Furthermore, in this procedure CD3⁺ lymphocytes (T-lymphocytes) are stimulated which require a previous contact with the antigen in order to be effective (acquired immunity). Natural killer cells however, as part of the innate immune response, require no previous contact with the antigen.

To date, no stably transfected cell line exists which serves as constant source of cytokines and may thus be used for the production of a medication for the treatment of tumor diseases in dogs.

### Aim

It is the aim of the present invention to eliminate the disadvantages of the state-of-the-art techniques and to provide cell lines for the permanent release of canine interleukines, which are suitable for the production of a medication for the tumor therapy.

### Solution

This aim is solved by claims of the present invention, by BHK Tet-On cells which are transfected with nucleic acid molecules comprising sequences coding for interleukin, so that interleukin is released after induction. In particular, BHK Tet-On cells are concerned which release canine interleukin-2 (cIL-2) and canine interleukin-12 (cIL-12), respectively, and which are suitable for the production of a medication for the tumor therapy.

The cell line which permanently releases canine interleukin is established from BHK Tet-On cells which are transfected with nucleic acid molecules comprising interleukin-coding sequences in such a way that these cells release interleukin after induction.

Using the release of cytokines 2 and 12, respectively, permanently lymphokin-activated killer cells are generated in a specific procedure, which are effective in the canine tumor therapy.

### Specific Features of the BHK Tet-On Cell Line

The present invention comprises cells, preferably BHK cells (Baby Hamster Kidney cells), which are transfected with gene sequences for canine interleukin-2 (cIL-2) and canine interleukin-12 (cIL-12), respectively, using the Tet-On system. These cells then permanently release canine interleukin-2 (cIL-2) and canine interleukin-12 (cIL-12), respectively, after stimulation with doxycycline.

The BHK cell lines originate from hamsters and are thus xenogenic. It is therefore guaranteed that these cell lines do not release any dog-specific proteins which might be interfering.

After transfection with the coding genes, preferably a nearly constant cytokine concentration of 5-50 ng interleukin/ml cell culture supernatant of canine interleukin-2 (cIL-2) and canine interleukin-12 (cIL-12), respectively, is reached in the cell culture. Natural killer cells, e.g. cells isolated from a tumor patient, are activated by interleukin 2 and/or 12 into lymphokin-activated killer cells (LAK) and are transferred into the patient as remedy for the treatment of tumor diseases.

The huge problem of state-of-the-art techniques for the treatment of tumor diseases, namely fluctuations of the cytokine concentrations due to injections of recombinant interleukin, is thus solved. Beyond this, considerably less side effects are to be expected in the patients.

The present invention furthermore includes the generation of lymphokin-activated killer cells (LAK) using isolated cells of tumor patients, preferably mammals, most preferably dogs, by permanent in vitro stimulation with canine IL-2 and IL-12, respectively, and thus the production of a suitable medication for the adoptive tumor immunotherapy.

One particular advantage of the present invention is that the patient is not subjected to any direct contact with genetically modified organisms, since the activation of lymphokin-activated killer cells with transfected BHK Tet-On cells is preferably performed outside the patient.

### EMBODIMENTS

### 1. Production of a Permanently Canine Interleukin (IL)-2 Producing Cell Line after Transfection of the Gene Coding for Canine IL-2 into the BHK Tet-On Cell Line

### 1.1. Cloning of IL-2

For the cloning of plasmid pTRUE-IL, cIL-2-mRNA is isolated from canine blood lymphocytes after previous stimulation of lymphocytes with concanavalin A. The purification of mRNA is performed according to the procedure of acidic guanidine isothiocyanate-phenol-choroform extraction with TRIzol® reagent (Invitrogen GmbH, Karlsruhe), e.g. described in CHOMCZYNSKI (1987), which is well known to experts skilled in the art.

Isolated cIL-2-mRNA is amplified via RT-PCR and transcribed into cDNA. For this purpose, e.g. the GeneAmp® RNA PCR Core Kit (Applied Biosystems) is used. The following primers are preferably used:

Forward-primer: 5′ TGA TGA GTG CAT TGG AG 3′
and
Reverse-primer: 5′ GAT TCT TTT TGT AAG CCC 3′.

Alternatively, the sequence of IL-2 is confirmed by sequencing. For this purpose, the amplified PCR-product is cloned into a suitable sequencing vector with standard procedures, e.g. the TOPO TA Cloning® Kit using the pCR®II-TOPO®-vector (both Invitrogen, Carlsbad, USA), and the correct sequence of the cloned IL-2 fragment is assessed with routine standard procedures.

The gene coding for cIL-2 is introduced into a Tet-On system, preferably cloned into vector pTRUE is part of the Tet-On system.

The commercially available vector pTRUE (Clontech) possesses one modification according to the present invention, since the originally present multiple cloning site (MCS) of pTRUE is exchanged for the MCS of plasmid pCITE (Novagene) which is also commercially available. Procedures concerning this cloning step are well known to those skilled in the art and disclosed in standard laboratory protocols.

Using PCR and the MCS of plasmid pCITE, recognition sequences for the restriction enzymes KpnI and MluI are introduced which are flanking the left and the right border of the cIL-2 gene in the vector pTRUE. For this purpose, preferably the following primers are used:

Forward-primer:
5′ GGT ACC ATG TAC AAA ATG CAA CTC TTG 3′
(the underlined sequence represents the
restriction site for KpnI)
and
Reverse-primer:
5′ ACG CGT CAA GTC AGT GTT GAG AAG ATG CTT TGA
CAA AAG G 3′
(the underlined sequence represents the
restriction site for MluI.

Vector pTRUE with the MCS of plasmid pCITE is cleaved with restriction enzymes KpnI and PstI, and the reporter gene luciferase is cleaved with restriction enzymes MluI and PstI. In a three-fold ligation, three fragments are fused to plasmid pTRUE-IL2 which is composed of the reporter gene luciferase, vector pTRUE with the MCS of plasmid pCITE, and the cIL-2 gene.

Seq. ID 1 shows the nucleic acid sequence of the cIL-2 gene

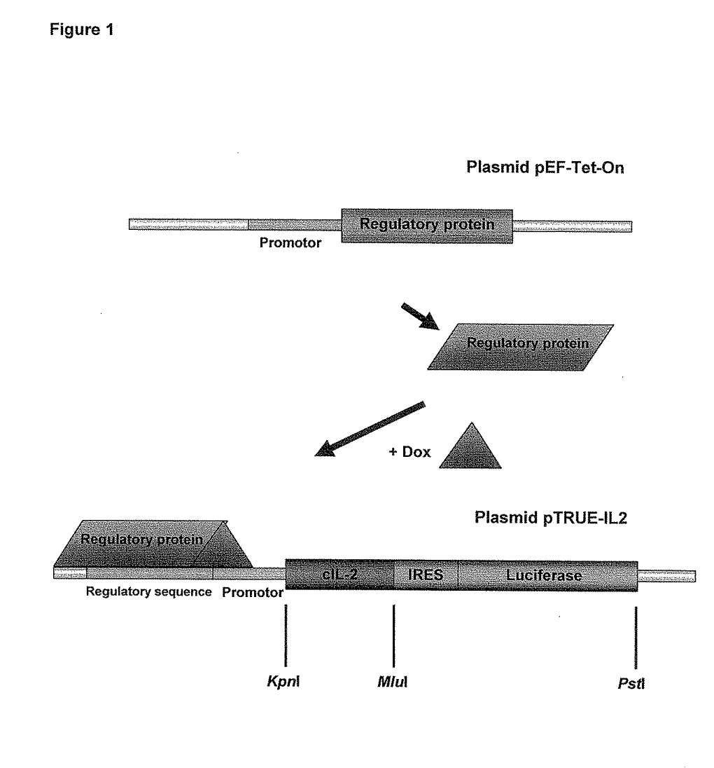

FIG. 1 gives a schematic representation of plasmid pTRUE-IL2.

Preferentially, an internal ribosomal entry site (IRES) is located immediately upstream of the reporter gene luciferase, allowing the translation of two single proteins, namely cIL-2 and luciferase.

Plasmid pTRUE-IL2 which is generated in this manner (FIG. 1) is linearized and then transferred via transfection into a BHK cell line which already harbors pEF-Tet-On (FIG. 1) stably integrated into its genome. The selection for successfully transfected cells is performed using the resistance genes for puromycin and G418.

It is obvious to experts in this field that the reporter gene luciferase is only used exemplarily and may either be replaced by further reporter genes such as GFP amongst others or alternatively be omitted completely.

Seq. ID1 shows the nucleic acid sequence of the cIL-2 gene in bold print. Underlined sequences represent the recognition sites for the restriction enzymes KpnI and MluI, normal print indicates flanking vector sequences.

Seq.ID 1
agctctcccatatggtcgacctgcaggcggccgcactagtgattggt
accatgtacaaaatgcaactcttgtcttgcatcgcactgacgcttgt
acttgtcgcaaacagtgcacctattacttcaagctctacaaaggaaa
cagagcaacagatggagcaattactgctggatttacagttgcttttg
aatggagttaataattatgagaacccccaactctccaggatgctcac
atttaagttttacacgcccaagaaggccacagaatttacacaccttc
aatgtctagcagaagaactcaaaaacctggaggaagtgctaggttta
cctcaaagcaaaaacgttcacttgacagacaccaaggaattaatcag
caatatgaatgtaacacttctgaaactaaagggatctgaaacaagtt
acaactgtgaatatgatgacgagacagcaaccattacagaatttctg
aacaaatggattaccttttgtcaaagcatcttctcaacactgacttg
acgcgtaatcccgcggccatggcggccgggagcatgcgacgtcgggc
ccaattcgccctatagtgagtcgtattacaattcac

### 1.2. Release of IL-2

BHK cells which are transfected with gene sequences for canine interleukin-2 (cIL-2) in the Tet-On system permanently release canine interleukin-2 (cIL-2) after stimulation with doxycycline.

For this purpose doxycycline, an inductor of the Tet-On expression system, is added to the cell culture medium to turn on the expression of proteins cIL2 and the reporter gene luciferase which were inserted by cloning. As cell culture medium, for example RPMI-medium is used. Alternatively, other media which are suitable for the cultivation of lymphocytes may be used as well, e.g. DMEM or Ham's F12.

The cell culture supernatants are testes for the content of biologically active cIL-2 in a suitable assay.

For that purpose, IL-2 dependent CTLL-2 cells (murine T cell tumor line) which proliferate in the presence of human as well as canine IL-2 are subjected to BHK supernatants for 72 h, and proliferation of these cells is subsequently verified in a MTT proliferation assay. Mitochondria of living cells metabolize MTT to formazane crystals whose concentration can be determined photometrically after dissolution of the crystals. As control and as well as IL-2-standard for the determination of the IL-2-concentration, serial IL-2 dilutions with defined rhlL-2 concentrations (50 ng IL-21 ml-0 ng IL-2/ml) are prepared which are similarly incubated with CTLL-2 cells. CTLL-2 cells show a considerable proliferation in the cIL-2-containing supernatants of transfected BHK cells, whereas BHK supernatants of both non-transfected and non-doxycycline-induced cell lines cause no CTLL-2 proliferation. Consequently, the cIL-2 which is secreted into the cell culture supernatant is biologically active.

### 2. Production of a Permanently Canine Interleukin (IL)-12 Producing Cell Line after Transfection of the Gene Coding for Canine IL-12 into the BHK Tet-On Cell Line

The cloning of plasmid pTRUE-IL-12 is performed via a combined integration of cDNA sequences coding for the canine IL-12 genes p35 and p40 (Büttner M, et al., 1998, Cytokine 10 (4), 241-8) into vector pTRE in such a way that these genes code for a rcscIL-12 (recombinant canine single chain) fusion protein.

The commercially available vector pTRUE (Clontech) possesses a modification according to the present invention, since the originally present multiple cloning site (MCS) of pTRUE is replaced by the MCS of the likewise commercially available plasmid pCITE (Novagene). Procedures concerning this cloning step are well known to those skilled in the art and disclosed in standard laboratory protocols.

For the cloning, both subunits of the canine IL-12, p35 and p40, are separately amplified by PCR. Preferably, the following primers are used for this amplification:

Forward-primer p40:
5′ggtaccatgcatcctcagcagttggtc 3′;
Reverse-primer p40:
5′cgtctcaggacaccgactcccgggacactgcaggacacagatgcc
cagtc3′;
Forward-primer p35:
5′cgtctcggtcccaggtgtcggtatgtgcccgccgcgcggcctcc 3′
Reverse-primer p35:
5′acgcgtttaggaagaattcagataactcatcattatatcgatggt
caccg 3′.

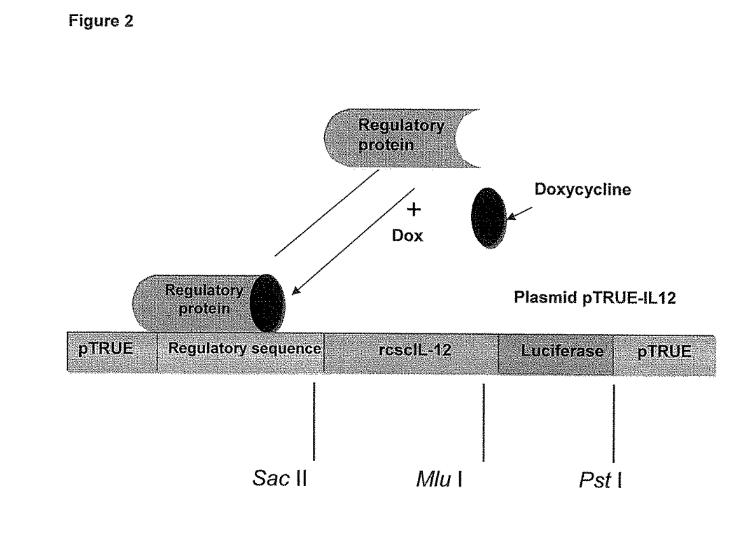

The PCR-amplification is performed according to procedures which are well known to experts skilled in the art, using cDNA of the canine genes p35 and p40 as disclosed by Büttner M. et al. (1998, Cytokine 10 (4), 241-8). A specific feature is that both cDNAs (for p35 and p40) are now joined by ligation to form one cDNA coding for the ORF (open reading frame) of a fusion protein. Between these two sequences, a sequence of 30 nucleotides is inserted by primer extension, a technique well known to experts in this field. This sequence codes for 10 amino acids of the bovine elastin and allows for a normal and undisturbed folding of the two sequences, thus ensuring the activity of the protein. FIG. 2 shows plasmid pTRUE-IL-12.

Seq. ID2 shows the nucleic acid sequence of plasmid pTRUE-IL-12, comprising the first 987 nucleotides of the p40 cDNA, then 30 nucleotides coding for 10 amino acids of the bovine elastin, followed by the nucleotides of p35 which code for the P35-protein. Sequencing and purification of the amplificates is performed according to standard protocols.

The amplificate of the joint sequence (Seq. ID2) is subsequently cloned into a commercially available pTRE-vector together with cDNA coding for luciferase. For this three-fold ligation, respective restrictions sites for SacII, MluI, and PstI as shown in FIG. 2 are used. Subsequently, commercially available BHK baby hamster kidney Tet-On cells are transfected via lipofection with the rcscIL-12-pTRE/luciferase vector according to the instructions of the manufacturer.

As selection marker for cloning, preferably puromycin is used. As cell culture medium, for example RPMI-medium is used. Alternatively, also other media may be used which are suitable for the cultivation of lymphocytes, e.g. DMEM or Ham's F12.

Cells with are successfully transfected with the rcscIL-12-pTRE/luciferase vector (FIG. 2) are tested in a luciferase assay and induced by addition of doxycycline to the RPMI medium.

The addition of doxycycline induces the expression of the canine IL-12 gene, and IL-12 is released into the culture supernatant after induction.

Seq. ID 2 shows the nucleic acid sequence of the IL-12 fusion protein. The fusion protein begins with P40 lacking the stop-codon, followed by 10 amino acids of the bovine elastin which are highlighted by an underlined section and the subsequent nucleic acid sequence of P35 protein which begins with the 26th amino acid (start with aag for arginine). The fusion protein ends with the stop-codon of the P35 protein, taa.

Seq.ID 2
atgcatcctcagcagttggtcatctcctggttttccctcgttttgct
ggcgtcttccctcatgaccatatgggaactggagaaagatgtttatg
ttgtagagttggactggcaccctgatgcccccggagaaatggtggtc
ctcacctgccatacccctgaagaagatgacatcacttggacctcagc
gcagagcagtgaagtcctaggttctggtaaaactctgaccatccaag
tcaaagaatttggagatgctggccagtatacctgccataaaggaggc
aaggttctgagccgctcactcctgttgattcacaaaaaagaagatgg
aatttggtccactgatatcttaaaggaacagaaagaatccaaaaata
agatctttctgaaatgtgaggcaaagaattattatggacgtttcaca
tgatggtggctgacggcaatcagtactgatttgaaattcagtgtcaa
aagtagcagaggcttctctgacccccaaggggtgacatgtggagcag
tgacactttcagcagagagggtcagagtggacaacagggattataag
aagtacacagtggagtgtcaggaaggcagtgcctgcccctctgccga
ggagagcctacccatcgaggtcgtggtggatgctattcacaagctca
agtatgaaaactacaccagcagcttcttcatcagagacatcatcaaa
ccagacccacccacaaacctgcagctgaagccattgaaaaattctcg
gcacgtggaggtcagctgggaataccccgacacctggagcaccccac
attcctacttctccatgacattttgcgtacaggcccagggcaagaac
aatagagaaaagaaagatagactctgcgtggacaagacctcagccaa
ggtcgtgtgccacaaggatgccaagatccgcgtgcaagcccgagacc
gctactatagttcatcctggagcgactgggcatctgtgtcatgcagt
gtcccgggagtaggtgtcccaggtgtcggtaggagcctacccacagc
ctcaccgagcccaggaatattccagtgcctcaaccactcccaaaacc
tgctgagagccgtcagcaacacgcttcagaaggccagacaaactcta
gattatattccctgoacttccgaagagattgatcatgaagatatcac
aaaggataaaaccagcacagtggaggcctgcttaccactggaattaa
ccatgaatgagagttgcctggcttccagagagatctctttgataact
aacgggagttgcctggcctctggaaaggcctcttttatgacggtcct
gtgccttagcagcatctatgaggacttgaagatgtaccagatggaat
tcaaggccatgaacgcaaagcttttaatggatcccaagaggcagatc
tttctggatcaaaacatgttgacagctatcgatgagctgttacaggc
cctgaatttcaacagtgtgactgtgocacagaaatcctcccttgaag
agccggatttttataaaactaaaatcaagctctgcatacttcttcat
gctttcagaattcgtgcggtgaccatcgatagaatgatgagttatct
gaattcttcctaa

### Detection of Bioactivity of IL-12 Released in the Supernatant

The bioactivity of IL-12 released by the BHK Tet-On cell line according to the present invention can for example be determined via a measurement of INF-gamma, since IFN-gamma is specifically synthesized only after stimulation of PMBCs (Peripheral Blood Mononuclear Cells) with IL-12 and can then by measured in a canine INF-gamma specific ELISA (R&D Systems) and compared with a standard control.

For this purpose, the PBMCs are incubated both with the supernatant of the IL-12 releasing cell line according to the present invention and in addition with the IL-12 of the standard control.

The results demonstrate that IL-12 which is released by the BHK Tet-On cell line according to the present invention and IL-12 of the standard solution are equally effective in inducing an INF-gamma synthesis.

The amount of IL-12 released is determined by incubation of PMBCs in addition with 1 μg/ml of an anti-canine IL-12 antibody (R&D Systems). The amount of IL-12 released is evidently strongly increased.

### 3. Synthesis of Lymphokin-Activated Killer Cells and Utilization of Lymphokin-Activated Killer Cells for the Production of a Medication for the Treatment of Tumors in Dogs

Natural killer cells (NK cells) belong to the lymphocytes and are able to recognize abnormal cells such as tumor cells via specific receptors and to eliminate these. Natural killer cells are activated by cytokines, e.g. IL-2 and IL-2, which are in vivo released by macrophages. After activation, natural killer cells produce large amounts of interferon-gamma (IFN-gamma).

The use of lymphokin-activated killer cells (LAK) which were already activated in vitro by interleukin represents a substantial progress in the tumor therapy of patients (mammals, especially dogs).

For this purpose, natural killer cells are isolated from a tumor patient and co-cultivated in a suitable medium over a period lasting between 7 days and 2 weeks with BHK Tet-On cell lines according to the present invention. These BHK Tet-On cells specifically release IL-2 and IL-12, respectively, in such a way that the NK cells turn into lymphokin-activated killer cells which are retransferred into the patient as medication for the treatment of tumor diseases.

As cell culture medium, for example RPMI-medium is used. Alternatively, also other media may be used which are suitable for the cultivation of lymphocytes, e.g. DMEM or Ham's F12.

In particular, natural killer cells which are isolated from the patient, preferably dogs, according to the standard protocol and BHK Tet-On cells according to the present investigation are co-cultivated in a bioreactor. Such bioreactors are for example well-established in the production of monoclonal antibodies and preferably possess two chambers, which are separated by a semi-permeable membrane. In this process, the cytokine-producing BHK Tet-On cell lines according to the present invention (IL-2 and/or IL-12) placed in one chamber and the NK cells, which were isolated from the dog, are placed in the other chamber to allow a free diffusion of the released interleukin through the membrane. Due to this continued release of interleukin, the natural killer cells are transformed into a state which resembles the in vivo state of lymphokin-activated killer cells (LAK) and can be utilized as medication of the treatment of malignant tumors.

In contrast to the injection of recombinant interleukin, the NK cells are stimulated more efficiently. After the desired incubation time (preferably 7 to 21 days), the stimulated NK cells are re-transferred into the patient by infusion. In addition to the process of stimulation which is similar to the process occurring in the body, the continuous synthesis of IL-2/IL-12 by the NK cells permanently provides new cytokine molecules and prevents a decrease in cytokine concentration.

Due to their expertise, physicians are able to decide which method is most suitable to return these IL-2 and/or IL-12 lymphokin-activated killer cells (LAK) to the patient, and which amount of cells should be used in order to achieve a therapeutically reasonable treatment of malignant tumors.

The lymphokin-activated killer cells (LAK) may thereby also be administered as part of a pharmaceutically acceptable composition or as prodrug.

According to the present invention, the term patient refers to human beings and vertebrates. The invention can thus be used in human and veterinary medicine. Particularly suitable is the application in dogs.

1. Cell line for the permanent release of interleukin, which is characterized in that BHK Tet-On cells are concerned which are transfected with nucleic acid molecules containing interleukin-coding sequences, so that interleukin is released after induction.

2. Cell line according to claim 1, which is characterized in that the interleukin-coding sequences code for interleukin 2 or interleukin 12.

3. Cell line according to claim 2, which is characterized in that the cell line releases 5-50 ng interleukin/ml cell culture supernatant.

4. Cell line according to claim 1, which is characterized in that the interleukin which is released is biologically active in the MTT proliferation assay.

5. Cell line according to claim 1, which is characterized in that the interleukin which is released is biologically active in the IFN-gamma assay.

6. Cell line according to claim 1, which is characterized in that it is co-cultivated with natural killer cells in such a way that lymphokin-activated killer cells are generated.

7. Procedure for the production of a permanently canine interleukin releasing cell line according to claim 1, which is characterized in that BHK Tet-On cells are transfected with nucleic acid molecules comprising interleukin-coding sequences, so that interleukin is released after induction.

8. Procedure according to claim 7, which is characterized in that the interleukin-coding sequences code for interleukin 2 or interleukin 12.

9. Procedure according to claim 8, which is characterized in that the sequences coding for interleukin 12 code for a fusion protein consisting of p35 and p40.

10. Procedure according to claim 7, which is characterized in that the induction is performed with doxycycline.

11. Utilization of a cell line according to claim 1 for the production of a medication for the treatment of tumor diseases.
